(19) United States
(12) Patent Application Publication
(10) Pub. No.: US 2006/0173080 A1
(43) Pub. Date: Aug. 3, 2006

(54) Methods of modulating TNF using bupropion

(75) Inventors: Eric Altschuler, New York, NY (US); Richard E. Kast, Burlington, VT (US)

Correspondence Address: BUCHANAN INGERSOLL LLP;(INCLUDING BURNS, DOANE, SWECKER & MATHIS), P.O. BOX 1404, ALEXANDRIA, VA, 22313-1404, US

(73) Assignee: The Regents of the University of California, Oakland, CA (US)

(21) Appl. No.: 11/291,730

(22) Filed: Dec. 1, 2005

Related U.S. Application Data
(63) Continuation-in-part of application No. 10/244,037, filed on Sep. 14, 2002, now abandoned.
(60) Provisional application No. 60/656,560, filed on Feb. 24, 2005.

(51) Int. Cl. A61K 31/137

(52) U.S. Cl. 514/649

(57) ABSTRACT

A new method employing a known compound, bupropion hydrochloride (.±.)-1-(3-chlorophenyl)-2-[(1,1-dimethylethyl)amino]-1-propanone hydrochloride, and its analogues, in a new use for the treatment of TNF-related disorders is described.

### CROSS REFERENCE TO RELATED APPLICATIONS

This application claims priority to U.S. Provisional Application Ser. No. 60/656,560 filed Feb. 24, 2005, and is a continuation-in-part of U.S. patent application Ser. No. 10/244,037, the disclosures of which are incorporated herein by reference in their entirety.

### FIELD OF THE INVENTION

The present invention generally concerns tumor necrosis factor (TNF) antagonists or TNF blockers for the treatment of autoimmnune diseases, inflammatory diseases, such as Crohn's, neurological disorders, trauma, injuries or compression, demyelinating neurological disorders, including multiple sclerosis; neurodegenerative diseases, including Alzheimer's disease; muscular disorders; and disorders of the optic nerve and retina (hereinafter “TNF-related Disorders”). More particularly, the small molecule TNF antagonists, TNF inhibitors or TNF blockers, are used for the treatment, prevention or amelioration of these TNF-related Disorders by modulating the action of TNF in the human body.

The present invention most particularly concerns a new method employing a known compound, bupropion hydrochloride (.±.)-1-(3-chlorophenyl)-2-[(1,1-dimethylethyl)amino]-1-propanone hydrochloride, and its analogues, in a new use for the treatment of these TNF-Related Disorders.

### BACKGROUND

Inflammatory Diseases

A number of inflammatory agents, e.g. bacteria, viruses, injury, etc., can cause Inflammation. Inflammation is promoted at a cellular level by these agents binding to the cell surface. Once inflamed, a cell releases pro-inflammatory messenger proteins called cytokines. An inflammatory cascade is initiated when these inflamed cells cause neighboring cells to become inflamed.

Tumor necrosis factor (TNF), is a naturally occurring cytokine that is involved in the development and progression of many inflammatory, infectious and autoimmune diseases. In rheumatoid arthritis (RA), TNF-alpha, a major player in the pathology of joints, stimulates bone and cartilage absorption, facilitates inflammation and inhibits bone formation. The immune system is finely balanced either directly by the activities of pro-inflammatory and anti-inflammatory mediators or indirectly by their ability to regulate the production of other immunoregulatory molecules. At normal levels, the protein is essential for effective immune function. Overproduction of TNF as a result of age, genetic and other influences, however, contributes to the pathology of numerous diseases. High levels of TNF-alpha are present in patients with both RA and Crohn's disease.

When an immunomodulatory drug is administered, it blocks the inflammatory signal initiated at the cell surface. This results in a down-regulation of the inflammatory cascade. TNF-alpha overproduction can be suppressed in two ways: (1) by inactivating circulating levels of TNF-alpha with antibodies, or soluble receptors; and (2) by interfering with synthesis or release of TNF-alpha from producing cells.

TNF is formed by the cleavage of a precursor transmembrane protein, forming soluble molecules that aggregate to form trimolecular complexes; These complexes then bind to receptors found on a variety of cells. Binding produces an array of pro-inflammatory effects, including release of other pro-inflammatory cytokines, including interleukin (IL)-6, IL-8, and IL-1; release of matrix metalloproteinases; and upregulation of the expression of endothelial adhesion molecules, further amplifying the inflammatory and immune cascade by attracting leukocytes into extravascular tissues. TNF is now well established as key in the pathogenesis of inflammatory, autoimmune diseases such as rheumatoid arthritis (RA) and Crohn's Disease.

Other autoimmune diseases include, without limitation, diabetes melitus, multiple sclerosis, premature ovarian failure, scleroderm, Sjogren's disease, lupus, vilelego, alopecia (baldness), polyglandular failure, Grave's disease, hypothyroidism, polymyosititis, pempligus, colititis, autoimmune hepatitis, hypopituitarism, myocardititis, Addison's disease, autoimmune skin diseases, uveititis, pernicious anemia, and hypoparathyroidism.

Relatively recently commercially available, specific inhibitors of TNF, provide the possibility of therapeutic intervention in TNF-mediated diseases. Some therapeutic success in treating Crohn's Disease, RA and Psoriatic Arthritis has been demonstrated with, chimeric anti-TNF monoclonal antibodies (e.g., infliximab), and recombinant fusion proteins (e.g., etanercept). The latter consisting of two soluble TNF receptors joined by the Fc fragment of a human IgG1 molecule. Other specific anti-TNF agents under development, including D2E7 (a human anti-TNF mAb), CDP 571 (a chimeric, but 95% humanized, anti-TNF mAb) and a pegylated soluble TNF type 1 receptor are also somewhat successful. Thalidomide and its functional analogues “SelCIDs” and “ImiDs” are anti-TNF agents recently used. None of these modalities has been very successful. There is a need for small, effective molecular TNF that would be non-toxic, stable, non-immunogenic and capable of passing the blood-brain barrier. Other innovative therapies might include gene therapy and the development of selective TNF production inhibitors, such as the TNF-converting enzyme.

Neurological Disorders

As with other organ systems, TNF has been shown to have a key role in the central nervous system. TNF-alpha, a protein previously considered to be only a component in the immune system, actually plays a key role in regulating neurotransmission in the central nervous system as well. It has long been recognized as being a key player in controlling cell death, but this new finding offers new insights into how cells interact within the human nervous system. This new role of TNF-alpha may provide researchers with possible new approaches to treating illnesses such as dementia, Alzheimer's disease, stroke, epilepsy and spinal cord injury.

Scientists have long believed that neurons were the really important cells in the nervous system because they control transmittal of signals within the entire CNS. Glial cells, comprising astrocytes, oligodendrocytes and microglia, were given credit only in a supportive role for the neurons, providing oxygen and nutrients to the neurons, shielding neurons from each other, and basically cleaning up dead neurons. New research, however, points to a much greater role for the glial cells—they can manufacture and release TNF-alpha into the CNS. The TNF-alpha has been shown to be able to regulate the expression of certain neurotransmitter receptors on the surface of neurons.

The binding of glutamate molecules from the fluid surrounding the cell to these receptors initiates signal production. When the glutamate and receptor meet, a nerve impulse, or signal, is produced. The more receptors present on the neuron surface, the more signals are produced. Normally, TNF-alpha is released as part of the inflammatory process following an injury to the cells. When TNF-alpha and glutamate are both present, cell-signaling activity seems to increase.

Because there is glutamate and TNF-alpha present in the spinal cord after injury, then perhaps TNF-alpha is actually enhancing the killing effect of the normal neurotransmitter. When nerve cells were exposed first to glutamate and then to TNF-alpha separately, neither had an impact on the normal killing rate. However, when the cells were exposed to even small amounts of both compounds, the killing effect increased by 120 percent.

Demyelinating disease neurological disorders (e.g. multiple sclerosis), immune disease, inflammation, trauma or compression, occur in different clinical forms, depending upon the anatomic site and the cause and natural history of the physiological problem. In Alzheimer's disease, for example, the brain undergoes serious neurodegeneration of unknown etiology. The fact that they can cause permanent neurological damage, that the damage can occur rapidly and be irreversible, and that current treatment of these conditions is unsatisfactory, is a common thread in all of these disorders.

Neurological conditions associated with TNF-alpha overproduction include, but are not limited to, acute spinal cord trauma, spinal cord compression, spinal cord hematoma, cord contusion, nerve compression, e.g., a herniated disc causing sciatic nerve compression, neuropathy, and pain. Also included are: cervical disc herniation, causing nerve compression in the neck; acute or chronic spinal cord compression from cancer metastasis; autoimmune disease of the nervous system; and demyelinating diseases, the most common condition being multiple sclerosis as stated hereinabove.

Treatment modalities employing steroid drugs such as cortisone to treat many of the aforementioned neurological problems and conditions are particularly hazardous because they are used either at high dosage, with a corresponding increasing risk of side effects, or because they are used chronically. Moreover, steroids are only partially effective or completely ineffective.

The ability of the body to repair injury to the nervous system is limited. The devastating nature of these diseases and the lack of effective therapy underscore the urgent need for early therapy to prevent or limit neuronal death. Therapies directed against TNF to dramatically limit inflammation by interrupting the inflammatory cascade at a fundamental level should be ideally suited. However, only very limited success has been achieved.

Therefore, there is a need for new TNF inhibitors therapeutically effective for a wide variety of neurological and related disorders. As stated hereinabove, these disorders are diverse. These further include Guillain Barre syndrome, and myasthenia gravis; degenerative disorders of the nervous system, including Parkinson's disease and Huntington's disease; disorders of related systems of the retina and of muscle, including optic neuritis, macular degeneration, diabetic retinopathy, dermatomyositis, amyotrophic lateral sclerosis, and muscular dystrophy; and injuries to the nervous system, including traumatic brain injury, acute spinal cord injury, and stroke.

There remains a need for a new pharmacologic treatment of these aforementioned physiological problems of the nervous system associated with autoimmune disease, demyelinating diseases, neurodegenerative diseases, trauma, injuries and compression with the pharmacological use of new TNF antagonists or TNF blockers that will be beneficial for the large number of patients afflicted. Drugs such as etanercept, infliximab, pegylated soluble TNF Receptor Type I (PEGs TNF-R1), other agents containing soluble TNF receptors, CDP571 (a humanized monoclonal anti-TNF-alpha antibodies), thalidomide, phosphodiesterase 4 (IV) inhibitor thalidomide analogues and other phosphodiesterase IV inhibitors are generally unsatisfactory because they provide either no remission, or only partial alleviation of suffering.

The current treatments with injectable proteins have risks and limitations. An orally active molecule that reduces TNF-/IL-1 synthesis could either replace the use of the injectables or provide better disease control when used in conjunction with other therapies. Additionally, several of these TNF agents will not cross the blood-brain barrier. It is painfully obvious that a small, effective, non-immunogenic molecule is needed.

Pharmacologic chemical substances, compounds and agents which are used for the treatment of neurological disorders, trauma, injuries and compression having various organic structures and metabolic functions have been disclosed in the prior art. For example, U.S. Pat. Nos. 5,756,482 and 5,574,022 to Roberts et al disclose methods of attenuating physical damage to the nervous system and to the spinal cord after injury using steroid hormones or steroid precursors such as pregnenolone, and pregnenolone sulfate in conjunction with a non-steroidal anti-inflammatory substance such as indomethacin. These patents do not teach the use of a TNF antagonist or TNF blocker for the suppression and inhibition of the action of TNF in the human body to treat TNF-related disorders, as in the present invention.

U.S. Pat. No. 5,605,690 to Jacobs discloses a method for treating TNF-dependent inflammatory diseases such as arthritis by administering to a human a TNF antagonist, such as soluble human TNFR, a peptide. This patent does not teach the use of a TNF antagonist or TNF blocker, as in the present invention.

U.S. Pat. No. 5,656,272 to Le et. al discloses methods of treating TNF-mediated Crohn's disease using chimeric anti-TNF antibodies. This prior art patent does not teach the use of a TNF antagonist or TNF blocker, as in the present invention.

U.S. Pat. No. 5,650,396 discloses a method of treating multiple sclerosis by blocking and inhibiting the action of TNF in a patient. This prior art patent does not teach the use of the TNF antagonist in the present invention.

U.S. Pat. No. 6,428,787, issued to Tobinick on Aug. 6, 2002, disclosed a myriad of molecular compounds purportedly effective in treating inflammatory diseases by inhibiting TNF-alpha, but there is no evidence that these are in any way effective in humans.

None of the above-identified references disclose or teach the use of TNF antagonists blockers of the present invention for inhibition TNF production in a human to treat TNF-related disorders, in which the TNF antagonist affords the patient complete remission or opportunity to heal, slows disease progression, prevents neurological damage, or otherwise improves the patient's life.

SUMMARY OF THE INVENTION

Provided herein are methods for inhibiting the action of TNF for treating various diseases associated with the presence of tumor necrosis factor. Accordingly, in one embodiment, a method of inhibiting a physiological activity of tumor necrosis factor (TNF) without causing beta-andrenergic receptor down-regulation, in a subject, is provided. The method includes administering to the subject an effective dose of bupropion hydrochloride ((+)-1-(3-chlorophenyl)-2-[(1,1-dimethylethyl)amino]-1-propanone hydrochloride), or analogues thereof. In some aspects, the physiological activity of TNF is associated with a disease, or symptom of a disease, such as Crohn's disease, rheumatoid arthritis, psoriasis, IgA nephropathy, anemia, myelodysplasia, appendicitis, peptic, gastric and duodenal ulcers, peritonitis, pancreatitis, pseudomembranous colitis, acute ulcerative colitis, chronic ulcerative colitis and ischemic colitis, diverticulitis, epiglottitis, achalasia, cholangitis, cholecystitis, hepatitis, nosicomial infection, inflammatory bowl disease, enteritis, Whipple's disease, diabetes, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pelvic inflammatory disease, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, urticaria, warts, wheals, vasulitis, cardiovascular disease, angiitis, endocarditis, arteritis, atherosclerosis, thrombophlebitis, pericarditis, myocarditis, myocardial ischemia, periarteritis nodosa, rheumatic fever, rheumatoid arthritis, Alzheimer's disease, coeliac disease, congestive heart failure, adult respiratory distress syndrome, meningitis, encephalitis, multiple sclerosis, cerebral infarction, cerebral embolism, Guillane-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, Sjogren's syndrome, myasthenia gravis, thryoiditis, systemic lupus erythematosus, lupus erythematosus, Addison's disease, pernicious anemia, Goodpasture's syndrome, Behcets's syndrome, allograft rejection, graft-versus-host disease, Type I diabetes, ankylosing spondylitis, Berger's disease, Type I diabetes, ankylosing spondylitis, spinal cord injury, Retier's syndrome, Graves disease, or Hodgkins disease.

In some embodiments, an effective dose of bupropion is in the range of 15 mg twice a day to 150 mg three times a day for an adult patient. However, it is understood that a clinician can easily determine the appropriate dosage for a particular subject. In general, the subject is a mammal, such as a mouse, a primate, or a human.

In another embodiment, a method for inhibiting the release of tumor necrosis factor (TNF) from a mammalian cell is provided. The method includes contacting the cell with bupropion hydrochloride ((+)-1-(3-chlorophenyl)-2-[(1,1-dimethylethyl)amino]-1-propanone hydrochloride), or analogues thereof, in an amount effective to inhibit the release of tumor necrosis factor (TNF). In some aspects, the cell is in a patient suffering from, or at risk for, a disease or disorder mediated by a tumor necrosis factor (TNF)-associated cascade.

In yet another embodiment, a method for decreasing the circulating level of tumor necrosis factor (TNF) in a subject is provided. The method includes administering to the subject an effective dose of bupropion hydrochloride ((+)-1-(3-chlorophenyl)-2-[(1,1-dimethylethyl)amino]-1-propanone hydrochloride), or analogues thereof, said administering being in an amount effective to decrease the circulating level of tumor necrosis factor (TNF) in the subject.

In another embodiment, a method for ameliorating symptoms resulting from a disorder associated with tumor necrosis factor (TNF) without causing beta-andrenergic receptor down-regulation, is provided. The method includes administering to the subject an effective dose of bupropion hydrochloride ((+)-1-(3-chlorophenyl)-2-[(1,1-dimethylethyl)amino]-1-propanone hydrochloride), or analogues thereof, said administering being in an amount effective to decrease the circulating level of tumor necrosis factor (TNF) in the subject thereby ameliorating the symptoms of the disorder.

In some embodiments, the bupropion hydrochloride ((+)-1-(3-chlorophenyl)-2-[(1,1 -dimethylethyl)amino]-1-propanone hydrochloride), or analogues thereof, is administered in conjunction with a steroid or an antibody directed against tumor necrosis factor (TNF).

In another embodiment, a method for treating tumor necrosis factor (TNF) associated diseases without causing beta-andrenergic receptor down-regulation is provided. The method includes administering bupropion hydrochloride ((+)-1-(3-chlorophenyl)-2-[(1,1-dimethylethyl)amino]-1-propanone hydrochloride),or analogues thereof, in an effective dose to alleviate or eliminate TNF associated disease symptoms and pathology; and monitoring the patient's response wherein the dose is adjusted according to degree of alleviation and elimination of the symptoms and pathology. In one aspect, the monitoring the patient's response includes monitoring the patient's Crohn's disease activity index (CDAI) level. In other aspects, the monitoring the patient's response includes monitoring the patient's circulating level of TNF.

In other embodiments, methods for treating rheumatoid arthritis, psoriasis, IgA nephropathy, anemia, myelodysplasia, neurological conditions, conditions of the optic nerve or retina, and muscular diseases in patients without causing beta-andrenergic receptor down-regulation, are provided. The methods include administering bupropion hydrochloride ((+)-1-(3-chlorophenyl)-2-[(1,1-dimethylethyl)amino]-1-propanone hydrochloride), or analogues thereof, in an effective dose to alleviate or eliminate symptoms and pathology; and monitoring the patient's response wherein the dose is adjusted according to degree of alleviation and elimination of symptoms. In the step of administering the TNF antagonist to a human, the TNF antagonist is performed through any of the following routes including subcutaneous, intravenous, intrathecal, intramuscular, intranasal oral, or transepidermal.

In other embodiments, the invention relates to a pharmaceutical composition comprising, as an active component, a compound of bupropion hydrochloride or its therapeutically effective analogue, together with a pharmaceutically acceptable excipient or carrier. The term “pharmaceutically acceptable” is intended to indicate that the excipient or carrier included in the composition is compatible with the other ingredients and not toxic or otherwise deleterious to a patient to whom the composition is administered. Accordingly, it is an object of the present invention to provide TNF antagonists for a new pharmacologic treatment of TNF-related disorders, such that the use of these TNT antagonists will result in significant amelioration of these conditions.

Other objects of the present invention include providing a TNF antagonist that reduces inflammation to a patient by inhibiting the action of TNF for the immediate, short term and long term, such that this reduction in inflammation will produce clinical improvement in the patient to heal, slow disease progression, prevent neurological damage, or otherwise improves the patient's life.

Another object of the present invention is to provide TNF antagonists that can offer acute and chronic treatment for neurological conditions caused by neurological trauma, compression, injury or disease; such conditions including acute spinal cord or brain injury, herniated nucleus pulposus, spinal cord compression due to metastatic cancer, primary or metastatic brain tumors, chronic pain syndromes due to metastatic tumor, increased intracranial pressure, demyelinating diseases such as multiple sclerosis, neurodegenerative diseases such as Alzheimer's disease, inflammatory CNS disease, such as subacute sclerosing panencephalitis, and other related neurological disorders and diseases.

Another object of the present invention is to provide a TNF antagonist that can offer acute and chronic treatment regimens for neurological and related diseases. Examples of diseases in these categories include but are not limited to diseases of the central and peripheral nervous system such as Parkinson's disease, Bell's palsy, Guillain-Barre syndrome.

Another object of the present invention is to provide a TNF antagonist that can offer acute and chronic treatment for retinal and neuro-ophthalmic diseases. Examples of diseases in these categories include but are not limited to optic neuritis, macular degeneration and diabetic retinopathy.

Another object of the present invention is to provide a TNF antagonist that can offer acute and chronic treatment for muscular diseases and diseases of the neuromuscular junction. Examples of diseases in these categories include but are not limited to dermatomyositis, amyotrophic lateral sclerosis and muscular dystrophy.

Another object of the present invention is to provide a TNF antagonist that can offer acute and chronic treatment regimens for degenerative neurological disorders and neurologic disorders of uncertain etiology. Examples of diseases in these categories include but are not limited to Alzheimer's disease, Huntington's disease and Creutzfeld-Jakob disease.

Another object of the present invention is to provide a TNF antagonist that can offer acute and chronic treatment regimens for neurologic injuries. Examples of diseases in these categories include but are not limited to acute spinal cord injury, acute brain injury and stroke.

Another object of the present invention is to provide a TNF antagonist that can offer acute and chronic treatment regimens for inflammatory and autoimmune disorders of the nervous system, examples being subacute sclerosing panencephalitis and myasthenia gravis.

Another object of the present invention is to provide a TNF antagonist that can offer acute and chronic treatment that is not hazardous to a patient.

Yet another object of the present invention is to provide a TNF antagonist that can offer acute and chronic treatment that is not prohibitively expensive.

The details of one or more embodiments of the disclosure are set forth in the accompanying drawings and the description below. Other features, objects, and advantages will be apparent from the description and drawings, and from the claims.

### DESCRIPTION OF THE DRAWINGS

FIG. 1 is a bar graph depicting TNF-α levels (top) and IL-1β levels in mice treated with a placebo or with bupropion hydrochloride ((+)-1-(3-chlorophenyl)-2-[(1,1-dimethylethyl)amino]-1-propanone hydrochloride) (WB).

FIG. 2 is a line graph depicting survival curves for mice treated with a placebo or with increasing amounts of bupropion hydrochloride ((+)-1-(3-chlorophenyl)-2-[(1,1-dimethylethyl)amino]-1-propanone hydrochloride) (WB).

FIG. 3 is a bar graph depicting IFN-γ levels in mice treated with a placebo or with increasing amounts of bupropion hydrochloride ((+)-1-(3-chlorophenyl)-2-[(1,1-dimethylethyl)amino]-1-propanone hydrochloride) (WB).

FIG. 4 is a line graph depicting Bupropion protection of mice against a lethal challenge of LPS. Male BALB/c mice were injected with 11, 33 or 100 mg/kg of bupropion or with placebo i.p. in saline 1 h before challenge with 600 μg of E. coli LPS (Sigma, St. Louis, Mo.) by i.p. route. Mice were sacrificed 90 min for detection of cytokines in the serum by sandwich ELISA. Values represent the means±SEM of 5 mice per group. * P<0.05.

FIG. 5 provides bar graphs depicting the effects of bupropion treatment in cytokine production in mice challenged with LPS. Groups of BALB/c mice were injected with bupropion or with placebo i.p. in saline 1 h before challenge with 600 μg of LPS (DL-100) by i.p. route. Bupropion significantly decreased levels of pro-inflammatory cytokines TNF, interleukin-1β (Il-1β), and interferon-gamma (INFγ), decreased with a trend toward significance the inflammatory mediator nitric oxide (NO), and had significantly increased levels of the anti-inflammatory (in this situation) Th2 cytokine Il-10. *P<0.05.

FIG. 6 is a bar graph depicting normalization of platelet and leukocyte levels by bupripion treatment. Groups of BALB/c mice were injected with bupropion 100 mg/kg (Wellbutrin, Glaxo) or with placebo i.p. in saline 1 h before challenge with 600 μg of LPS (DL-100) by i.p. route. Platelet and leukocyte numbers were evaluated in peripheral blood samples collected 4 h after LPS challenge in an automated cell counter (Bayer). * P<0.05 placebo compared with normal levels; # P<0.05 bupropion compared with placebo. In a shock often platelet levels drop precipitously due to intravascular coagulation and leukocyte also fall secondary to TNF induced apoptosis. The normalization by bupropion is consistent with the other effects of bupropion in this model.

FIG. 7 is a table providing the results of the survival rates for animals treated with bupropion.

The invention is further described in the following general procedures, preparations and examples that are not in any way intended to limit the scope of the invention as claimed.

### DETAILED DESCRIPTION

Cytokines and growth factors, the cellular mediators of the immune system, have profound effects on disease processes. The immune system is finely balanced either directly by the activities of pro-inflammatory and anti-inflammatory mediators or indirectly by their ability to regulate the production of other immunoregulatory molecules. Unregulated activities of these mediators can lead to the development of serious inflammatory and other diseases. Enhanced tumor necrosis factor-(TNF-) and interleukin-1 (IL-1) levels are associated with the development of rheumatoid arthritis, psoriatic arthritis and inflammatory bowel disease.

Provided herein are methods of ameliorating TNF-associated diseases by administering an effective amount of a pharmaceutical composition that includes bupropion. Pharmaceutical compositions of the invention may be in unit dosage form such as tablets, pills, capsules, powders, granules, elixirs, syrups, emulsions, ampoules, suppositories or parenteral solutions or suspensions for oral, parenteral, opthalmic, transdermal, intra-articular, topical, pulmonal, nasal, buccal or rectal administration or in any other manner appropriate for the formulation of anti-inflammatory compounds and in accordance with accepted practices such as those disclosed in Remington: The Science and Practice of Pharmacy. 19.sup.th Ed., Mack Publishing Company, 1995. The term “unit dosage” is intended to indicate a unitary, i.e. a single dose which is capable of being administered to a patient, and which may be readily handled and packed, remaining as a physically and chemically stable unit dose comprising either the active component as such or a mixture of it with solid or liquid pharmaceutical excipients or carriers. In the composition of the invention, the active component may be present in an amount of from about 0.1-100% by weight of the composition.

For oral administration in the form of a tablet or capsule bupropion hydrochloride or its therapeutically effective analogue may suitably be combined with an oral non-toxic, pharmaceutically acceptable carrier such as ethanol, glycerol, water or the like. Furthermore, suitable binders, lubricants, disintegrating agents, flavouring agents and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes or the like. Lubricants include, e.g., sodium oleate, sodium stearate, magnesium stearate, sodium benzoate, sodium acetate, sodium chloride or the like. Disintegrating agents include, e.g., starch, methylcellulose, agar, bentonite, xanthan gum or the like.

For the preparation of solid compositions such as tablets, the active compound of bupropion hydrochloride or its therapeutically effective analogue is mixed with one or more excipients, such as the ones described above, and other pharmaceutical diluents such as water to make a solid preformulation composition containing a homogenous mixture. The term “homogenous” is understood to mean that the compound of bupropion hydrochloride or its therapeutically effective analogue is dispersed evenly throughout the composition so that the composition may readily be subdivided into equally effective unit dosage forms such as tablets or capsules.

Liquid formulations for either oral or parenteral administration of the compound of the invention include, for example, aqueous solutions, syrups, aqueous or oil suspensions and emulsion with edible oils such as cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose or polyvinylpyrolidone.

For parenteral administration, for example, intramuscular, intraperitoneal, subcutaneous or intravenous injection or infusion, the pharmaceutical composition preferably comprises a compound of bupropion hydrochloride or its therapeutically effective analogue dissolved or solubilised in an appropriate, pharmaceutically acceptable solvent. For parenteral administration, the composition of the invention may include a sterile aqueous or non-aqueous solvent, in particular water, isotonic saline, isotonic glucose solution, buffer solution or other solvent conventionally used for parenteral administration of therapeutically active substances, in particular antiproliferative agents. The composition may be sterilized by, for instance, filtration through a bacteria-retaining filter, addition of a sterilizing agent to the composition, irradiation of the composition, or heating the composition. Alternatively, the compound of the invention may be provided as a sterile, solid preparation, for example, a freeze-dried powder that is dissolved in sterile solvent immediately prior to use.

The composition intended for parenteral administration may additionally comprise conventional additives such as stabilizers, buffers or preservatives, for example, antioxidants such as methylhydroxybenzoate or the like. Compositions for rectal administration may be in the form of a suppository incorporating the active ingredient and a carrier such as cocoa butter, or in the form of an enema.

Compositions suitable for intra-articular administration may be in the form of a sterile aqueous preparation of the active ingredient, bupropion hydrochloride or its therapeutically effective analogue, which may be in microcrystalline form, for example, in the form of an aqueous microcrystalline suspension. Liposomal formulations or biodegradable polymer systems may also be used to present the active ingredient for both intra-articular and ophthalmic administration.

Compositions suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. For topical administration, the active component preferably comprises from 1% to 20% by weight of the composition, but the active ingredient may comprise as much as 50% w/w.

Compositions suitable for administration of bupropion hydrochloride or its therapeutically effective analogue to the nasal or buccal cavity or for inhalation include powder, self-propelling and spray formulations, such as aerosols and atomizers. Compositions suitable for nasal or buccal administration may comprise 0.1% to 20% w/w. for example about 2% w/w of active ingredient.

The composition may additionally comprise one or more other active components conventionally used in the treatment of various inflammatory diseases and conditions. Examples of such additional active components may be selected from the group consisting of glucocorticoids, vitamin D and vitamin D analogues, antihistamines, platelet activating factor (PAF) antagonists, anticholinergic agents, methylxanthines, .beta.-adrenergic agents, COX-2 inhibitors, salicylates, infomethacin, flufenamate, naproxen, timegadine, gold salts, penicillamine, serum cholesterol lowering agents, retinoids, zinc salts and salicylazosulfapyridine.

A suitable dosage of the compound of the invention will depend, inter alia, on the particular compound selected for the treatment, the route of administration, the age and condition of the patient, the severity of the disease to be treated and other factors well known to the practicing physician. The compound may be administered either orally or parenterally according to different dosing schedules, for example, daily or with weekly intervals. In general, a single dose will be in the range from 0.01 to 500 mg/kg body weight, such as from 0.1 to 150 mg/kg body weight. The compound may be administered as a bolus or in divided doses two or more times a day.

Crohn's Disease

Crohn's disease is a chronic and serious inflammatory disease of the gastrointestinal that eventually breaks down the intestinal wall. It afflicts more than 500,000 Americans, most of them under age 30. People with Crohn's disease may experience a number of symptoms including diarrhea, abdominal cramps and pain, fever, rectal bleeding, loss of appetite, and weight loss. The range and frequency of these symptoms varies. About half of all Crohn's patients at some point require surgery to remove intestinal obstructions or repair holes in the bowel. Crohn's disease is also sometimes referred to as ileitis, regional enteritis or colitis. Although Crohn's disease is the general term used to describe the condition, doctors use these specific terms to differentiate the portion of the bowel that is involved.

The complications that patients of Crohn's disease depends upon the location and severity of the disease. Serious complications include the swelling and scarring associated with Crohn's disease that can obstruct the intestine. When this occurs, a patient may experience painful cramps, or vomiting due to food that is not able to bypass the obstruction. Patients who experience an obstructive episode are usually hospitalized and given intravenous fluids and salts to compensate for fluid losses. Ulcers caused by Crohn's disease may channel through the gut wall to adjacent organs. Fistulas tracking into the abdominal cavity may produce infections and form abscesses. And, if a fistula fails to drain, abscesses may occur in the pelvis or the perineum. These may require surgery, and are treated usually treated with antibiotics to reduce the risk of further infection.

Rarely, penetrating ulcers tunnel through the layers of the gut into the abdominal cavity. This free perforation manifests in sudden abdominal pain, shock and excessive tenderness. The resulting peritonitis requires emergency surgery to seal the leak and clean the abdominal cavity to stop further infection. In other rare cases, a bleeding ulcer may burrow through the gut wall and shear an artery, producing life-threatening hemorrhaging and requiring emergency surgery.

Chronic complications associated with Crohn's Disease include anemia, malnutrition and growth retardation. Anemia may be the result of chronic blood loss, bone marrow depression, or failure to absorb iron and folic acid, all important in stimulating the production of red blood cells. Certain treatment medications, such as sulfasalazine, may exacerbate anemia, requiring diet supplementation. Loss of protein, tissue breakdown, poor diet and failure to absorb nutrients, may result in nutritional deficiencies and weight loss. About one-third of children with Crohn's experience growth retardation. Treatment

The etiology of Crohn's disease has not yet been discovered. Presently, treatment goals are to suppress inflammation, facilitate healing; and relieve the symptoms. It is a chronic inflammatory disease of the bowel with overactivity of the immune cells within the bowel, such as macrophages, lymphocytes and neutrophils that reside in the wall just below the lining or mucosal surface. If the intestinal mucosa is breached by bacteria, such as E.coli or Salmonella, these cells isolate and kill the bacteria. Under normal circumstances, the bowel returns to normal.

Sulfasalazine and mesalamine have shown efficacy in treating patients with mild to moderately active Crohn's. Some of these medications are available in enema or suppository form to target specific levels of the bowel. Antibiotics are used to control symptoms involving the colon, distal small bowel and perianal region. The antibiotics decrease the concentration of the bacteria residing in the bowel diminishing concentration of the breakdown products released when they die. These breakdown products may contribute to the inflammation associated with Crohn's disease. Side effects of the treatments include nausea, a metallic taste and peripheral neuropathy characterized as numbness in the hands and feet, resolving when the medication is discontinued. These treatments are expensive.

Corticosteroids are potent immunosuppressive medications used to treat inflammation in active Crohn's disease. Preparations available for oral, rectal and intravenous administration, work quickly and are fairly inexpensive. Unfortunately, when used long term they have many undesirable side effects, including adrenal gland suppression, hypertension diabetes, osteoporosis cataracts and hip fracture. Entocort EC capsules, formulated to release budesonide once they reach the intestine, is purported to have fewer side effects than other steroids, such as prednisone. About half of patients taking Entocort EC experienced improvement in their symptoms after two months of therapy.

Azathioprine, 6-mercaptopurine and Methotrexate are immunosuppressive medications used when inflammation cannot be controlled with corticosteroids. As with all immunosuppressive medications, these drugs have substantial side effects.

Infliximab, a monoclonal antibody, was released by the U.S. Food and Drug Administration in August 1998 to treat patients with moderate to severe Crohn's disease that is resistant to management with the immunosuppressors and medications above. Specifically acting against TNF-alpha, it has been studied in two randomized-controlled trials involving approximately 100 patients with Crohn's disease. Four weeks after receiving the medication, 50 to 81 percent of the patients showed signs of improvement and 33 percent went into remission, while 17 percent in the placebo group improved and 4 percent entered remission. In the second study, patients with active fistulas received infliximab or placebo on three occasions, two weeks apart. There was a 50 percent reduction in fistula drainage in nearly two-thirds of patients who received infliximab, and over half of the patients'fistulas closed. Although significant, the results are not particularly appreciated, especially by the great number of patients not responding.

The anti-TNF-alpha therapy is contraindicated in patients with an active infection and in patients with a history of malignancy because of the role that TNF-plays in combating the development and spread of cancer. Allergy to the medication and pregnancy are other contraindications. Patients with marked narrowing of the bowel should not receive this medication, because rapid healing may lead to scar tissue formation, resulting in complete bowel obstruction. Finally, anti TNF-alpha therapy is expensive, and no data is available on its long term use.

When the Food and Drug Administration gave early approval to the new drug Remicaid a little more than a year ago, it offered hope particularly to patients with the worst type of Crohn's —those with ulcers, or fistulas, that tunnel through the bowel wall into nearby organs or through the surface of the skin. In one study, half of the fistulas closed in 68 percent of patients taking Remicaid. In another, 48 percent of patients showed complete remission. Yet Remicaid has big risks. Such immunosuppression has been linked to lymphomas and other cancers. The FDA, ending the Remicaid trials after only three years in order to get the drug to desperately ill patients, closed the window on evaluating the drug's long-term effects.

Isis Pharmaceuticals announced that it has initiated Phase I clinical studies of ISIS 104838, a novel antisense inhibitor of TNT-alpha employing second-generation antisense chemistry, to treat inflammatory and autoimmune diseases such as rheumatoid arthritis (RA) and Crohn's. It will investigate the safety and efficacy of the drug administered intravenously (IV) and subcutaneously. Oral formulations are being developed in parallel.

Celgene has identified this route as being accessible through its two classes of small molecule, orally available agents: “SelCIDS” and “ImiDs”. These agents use different biochemical mechanisms to target underlying TNF-alpha overproduction without affecting general immune system function. However the results are not encouraging.

A monoclonal anti-TNF antibody has been found of some efficacy in CD, for example, in closing fistulas (Present D H, Rutgeerts P, Targan S. Hanauer S B, Mayer L, van Hogezand R A, Podolsky D K, Sands B E, Braakman T, De Woody K L, Schaible T F, van Deventer S J. Infliximab for the treatment of fistulas in patients with Crohn's disease. N Engl J Med 1999;340:1398-1405). However, the antibody is expensive, it must be given intravenously, and although not common, there is potential for severe side effects. Also, long-term effects of repeated treatments with anti-TNF antibody are unknown. In CD studies, even in patients who benefit from a given medicine, frequently, enough CD activity remains to significantly lower quality of life even though the Crohn's Disease Activity Index (CDAI) (Best W R, Becktel J M, Singleton J W. Rederived values of the eight co-efficients of the Crohn's Disease Activity Index (CDAI). Gastroenterology 1979,77:843-846) is under the somewhat arbitrary cutoff of 150 used to define remission. Clearly, easing of disease burden is of great value, but remission of all signs and symptoms of disease remains our goal.

Agonist binding to the beta-adrenergic receptor increases intracellular cAMP (Talmadge J, Scott R, Castelli P, Newman-Tarr T, Lee J. Molecular pharmacology of the .beta.-adrenergic receptor on THP-1 cells. Int J Immunopharmacol 1993;15:219-228). cAMP is believed to be a key intracellular regulator of TNF, and increases of cAMP from any origin have been shown to decrease TNF in a variety of in vitro and in vivo situations in animals and humans (Prabhakar U, Lipshutz D, Bartus J O, Slivjak M J, Smith E F 3rd, Lee J C, Esser K M. Characterization of cAMP dependent inhibition of LPS induced TNF production by rolipram, a specific phosphodiesterase IV inhibitor. Int J Immunopharmacol 1994.16:805-816; Guirao X, Kumar A, Katz J, Smith M, Lin E, Keogh C, Calvano S E, Lowry S F. Catecholamines increase monocyte TNF receptors and inhibit TNF through-2 adrenoreceptor activation. Am J Physiol 1997;273:E1203-1208; Sommer N. Loschmann P A, Northoff G H, et al. The antidepressant rolipram suppresses cytokine production and prevents autoimmune encephalitis. Nat Med 1995;1:244-248). Phosphodiesterases mediate a step in CAMP catabolism. Phosphodiesterase inhibitors increase cAMP levels and lower TNF levels. The phosphodiesterase inhibitor rolipram has been shown in different experimental models to not only lower TNF levels, but also be effective in mitigating disease severity in several animal models of human inflammatory/autoimmune disease (e.g., Sommer et al.). Recently, the .beta.-adrenergic agonist isoproteronol has been found to lower TNF levels when used into humans (Goebel M U, Mills P J, Irwin M R, Ziegler M G. Interleukin-6 and tumor necrosis factor-alpha production after acute psychological stress, exercise, and infused isoproterenol: differential effects and pathways. Psychosom Med 2000;62:591-598).

Similarly, it has been suggested that cases of CD remission seen after phenelzine treatment of depression (Kast R E. Crohn's disease remission with phenelzine. Gastroenterology 1998; 15:1034-1035) may be driven by increased cAMP-associated TNF decreases. Consistent with this notion, in a fascinating recent abstract it has been found that the levels of norepinephine were lower in gut mucosa in CD patients than in healthy controls or ulcerative colitis (UC) patients, and dopamine levels were lower in gut mucosa in CD and UC patients than in controls (Magro F, Vieira-Coelho M A, Fraga S, Serrao M P, Tavarela-Veloso F, Tome-Ribeiro, Soares-da-Silva P. Impaired synthesis or cellular storage of norepinephrine, dopamine and 5-hydroxtryptamine in inflammatory bowel disease (abstr). Am J Gastroenterol 2000;95:2556). Also we note that, amazingly, an MAO-I (Leib J. Remission of rheumatoid arthritis and other disorders of immunity in patients taking monoamine oxidase inhibitors. Int J Immunopharmacol 1983;5:353-357) has been reported to induce remission in rheumatoid arthritis, and we have suggested that increased monoamine-mediated TNF decrease is the mechanism behind this observation as well (Altschuler EL. Monoamine oxidase inhibitors in rheumatoid arthritis-anti-tumor necrosis factor, Int J Immunopharmacol 2000;22:1007-1008).

Given the long-term experience with and safety of phenelzine, further study of phenelzine in CD might be considered. However, phenelzine carries some risks: hypertensive crisis associated with dietary restriction breaches can be serious, and fatal hypertensive crises are seen with multiple medicine incompatibilities (e.g., meperidine). Other medicines that can increase monoaminergic tone without these risks could be considered. Bupropion hydrochloride is an antidepressant without the risks of MAOIs. The mechanism of bupropion's antidepressant action is unclear, but might involve weak inhibition of catecholamine re-uptake (Sanchez C, Hyttel J. Comparison of the effects of antidepressants and their metabolites on reuptake of biogenic amines and on receptor binding. Cell Mol Neurobiol 1999;19:467-489). Bupropion seems to be unique in that it is the only modem antidepressant that does not cause compensatory .beta.-adrenergic receptor down-regulation (Garrcha G, Smokcum R W, Stephenson J D, et al. Effect of some atypical antidepressants on .beta.-adrenoreceptor binding and adenylate cyclase activity in the rat forebrain. Eur J Pharmacol 1985;108:1-7).

Provided herein are treatments aimed at alleviating the symptoms associated with increased levels of TNF, such as Crohn's Disease, in patients that have failed to respond to other treatments. Also, multiple toxicity issues are associated with taking medicines such as azathioprine, methotrexate, and steroids over time (Sartor R B. New therapeutic approaches to Crohn;s disease. N Engl J Med 2000;342:1664-1666). In CD, TNF is increased, and TNF is thought to play a central role in CD pathogenesis (Papadakis K A, Targan S R. Role of cytokines in the pathogenesis of inflammatory bowel disease. Annu Rev Med 2000;51:289-298). Monoaminergic and dopaminergic treatments can lower levels of TNF-alpha via increased intracellular adenosine 3′,5′-cyclic monophosphate (cAMP). Data provided herein describe two cases in which bupropion hydrochloride has produced near complete and long-lasting (over 2 years) remissions of CD without significant side effects or toxicities.

A 44-year-old woman presented in May of 1999 for treatment of an episode of major depression, superimposed on a chronic mild depressed state (dysthymia). She had a 10-year history of active CD, radiographically limited to the colon, with a history of 1 resection of a very small section of large bowel. She was not usually pain-free during the week, though usually did have several pain-free hours in a given day. Overt blood was noted in the stool frequently. Inadvertent loss of stool several times a month required daily use of adult protective pads. She was taking fluoxetine 40 mg every day for depression, and mesalamine 500 mg twice a day. CDAI was 202. She had a history of approximately 1 CD flare per year requiring steroids.

After careful review of treatment options, she was started on bupropion (fluoxetine was stopped) with up-titration to 150 mg twice a day. Her abdominal complaints eased over the next months, and the major depression remitted. The baseline dysthymia remained. To better treat the dysthymia, bupropion was raised to 150 mg three times daily with a further lowering of her CDAI. The mesalamine was tapered off. Her CDAI is currently approximately zero. She has one well-formed bowel movement daily and has been without abdominal symptoms or encopresis for 19 months now. She has had no CD flares since starting bupropion. Twice in the last year, she stopped bupropion on her own accord because she felt completely well and felt she didn't need bupropion anymore. Abdominal cramping, and blood per rectum recurred on both occasions, but remitted within weeks of restarting bupropion.

The above-described response to bupropion was consistent with other responses in CD patients. For example, a 45-year-old man with a 20-year history of Crohn's enteritis with multiple surgeries, including 4 small bowel resections. In April of 1999, even though he was on azathioprine, the patient had a CD flare requiring bowel resection. After resolution of the flare, a retrospective history shows CDAI of approximately 275. The patient was taking fluoxetine to help with pain control. Because this was not effective and also because the patient was a smoker, it was suggested to the patient that he consider switching from fluoxetine to bupropion, and he did so. Even though he was not able to stop smoking, the patient felt better on bupropion so he decided to continue this medicine with his family doctor's consent. Currently, the patient is taking bupropion 150 mg three times daily. His Crohn's symptoms were alleviated completely, and his CDAI is about 45 as a result of 3-4 diarrheas a day, possibly secondary to no longer having an ileal-cecal valve. He has had no CD flares or associated surgeries since starting bupropion. Under his doctor's supervision, he has recently tapered his azathioprine from 100 to 50 mg every day without any increase in CDAI.

These cases indicate that bupropion can be used to treat additional TNF-associated diseases. Also, based upon this information physicians can consider the “TNF implications” for other medications that a patient may be using to treat, for example, CD.

Multiple Sclerosis

The inflammatory Cytokine Tumor Necrosis Factor-alpha (TNF-alpha) has been implicated as a mediator of oligodendrocyte (OL) cell injury. TNF-alpha is detectable within MS lesions and induces apoptosis of mature human OLs in vitro. One possible mechanism by which TNF-alpha mediates cell death is through the activation of c-jun N-terminal kinase (JNK). It has been shown that treatment of human OLs with TNF-alpha leads to activation of JNK. p53, a regulator of the cell cycle and apoptosis, is a mediator of TNF-alpha-induced apoptosis of OLs. In adult human OLs, p53 levels increased within 24 h after TNF-alpha treatment (100 ng/ml). The induced p53 was immunolocalized to the nucleus prior to the appearance of significant numbers of apoptotic cells. Overexpression of p53 by Adenovirus-mediated gene transfer into human OLs in vitro resulted in marked apoptosis as revealed by in situ cleavage of DNA (TUNEL positive), decreased Mitochondrial function, and release of Lactate Dehydrogenase into the culture medium. These in vitro studies demonstrated that increased p53 levels are associated with Apoptosis of human OLs. The findings further implicated p53 as a target for the JNK pathway activated during TNF-alpha-mediated cell death of human adult OLs. Clearly, Buproprion should be considered in these cases.

Glaucoma

About three million adult Americans suffer from glaucoma, making it one of the leading causes of blindness. Chronic glaucoma accounts for 90% of cases in the United States, usually appears in middle age and seems to have a genetic component. One out of five sufferers has a close relative with the condition. Doctors often refer to chronic glaucoma as the “sneak thief in the night” because it comes on gradually to steal one's vision. It may be well established before you notice the warning signs, e.g., the need for new glasses, loss of side vision, blank spots in your vision.

Other forms of glaucoma are less common but not less serious. Sudden, severe pain in the eyes, blurred vision and dilated pupils, sometimes with nausea or vomiting, may be an attack of acute, or narrow-angle, glaucoma. This type accounts for less than 10% of reported cases, but it comes on quickly and requires urgent medical attention. If left untreated, it can irreversibly damage the optic nerve, which carries visual images from the eye to the brain, causing blindness, sometimes in a matter of days.

Secondary glaucoma is usually associated with another eye disease or disorder, such as an enlarged cataract, uveitis (an inner-eye inflammation), an eye tumor or an eye injury. People suffering from diabetes are also susceptible to neovascular glaucoma, a particularly severe form of the disease. Congenital glaucoma is an extremely rare condition, affecting babies.

The eye's lens, iris and cornea are continuously bathed and nourished by a water-based fluid called aqueous humor. As new fluid is produced by cells inside the eye, excess fluid normally drains out through a complex network of tissue called the drainage angle, where the cornea and iris meet. An imbalance between the rate of production of aqueous humor and the rate of drainage will bring on chronic, or open-angle, glaucoma. This is the most prevalent form of the ailment and generally develops slowly with age.

Some people apparently inherit a condition of the inner eye in which the iris can block normal drainage channels. When this happens, the fluid does not drain out of the eye fast enough, and the sudden pressure from fluid buildup causes acute glaucoma. In newborns, defects in the drainage angle are the cause of congenital glaucoma. Both conditions need prompt medical attention to prevent potential loss of sight.

Raised intraocular pressure is one predisposing factor, but a significant portion of patients with glaucoma have normal intraocular pressure. In patients with glaucoma, and especially those with normal pressure glaucoma (NPG), it has been found that levels of the pro-inflammatory cytokine tumor necrosis factor-alpha (TNF) are elevated. Also, one gene which is mutated in autosomal dominant primary open angle glaucoma, in patients predominantly with normal or only moderately elevated intraocular pressure, seems to be associated with the TNF signaling pathway and pathogenic mutations may predispose patients to TNF associated apoptosis (Rezaie T, Child A, Hitchings R, et al. Adult-onset primary open-angle glaucoma caused by mutations in optineurin. Science 2002; 295: 1077-1079). Thus, a drug that could lower TNF may be beneficial in NPG, and we suggest that bupropion might be such a drug.

Congestive Heart Disease

Congestive heart failure is a disease of epidemic proportions with 4.6 million patients in the U.S. and in nearly half of these patients, the etiology of their disease is unknown. It is generally viewed as a progressive disease in which initial myocardial damage is followed by cardiac remodeling and progressive dilation of the left ventricle. Cardiac compensation is followed by progressive de-compensation and patients present with worsening symptoms including fatigue, shortness of breath, and edema. Recent investigation has focused on the role of the proinflammatory cytokine TNF-in the development of heart failure and in particular in the transition from compensated to decompensated heart failure. Although TNF-can modulate the function of a group of potentially important cardiac proteins, recent evidence suggests that TNF-modulates the expression of the two families of proteins that regulate the homeostatic balance within the extracellular matrix, the matrix metalloproteinases (MMPs) and the tissue inhibitors of metalloproteinases (TIMPs). Furthermore, transgenic mice harboring cardiac restricted overexpression of TNF-develop matrix remodeling, progressive fibrosis, and collagen denaturation that are associated with up-regulation of MMP activity. Anti-TNF-therapy using adenovirus-mediated gene therapy significantly inhibited MMP activity, prevented myocardial fibrosis and collagen denaturation. These preliminary studies led us to hypothesize that the development of end-stage heart failure is due in large part to cytokine-induced matrix remodeling and fibrosis, and that the development of fibrosis marks the irreversibility of the disease.

The present studies have identified three patients with congestive heart failure (CHF) whose TNF-alpha dropped profoundly on bupropion. A 68 year old woman with NYSHA Class I CHF who started on bupropion for major depression. Her TNF before bupropion was 347 pg/ml and 5 pg/ml after two months on bupropion 150 mg twice a day (BID). Her depression was partially controlled on bupropion.

In addition, a 40 year old gentleman with a history of two myocardial infarctions and NYSHA Class II CHF, who started on bupropion for major depression and desire to stop a 1.5 container/day chewing tobacco habit. His TNF before starting bupropion was 433 pg/ml, and pg/ml following up titration and then four weeks on bupropion 150 mg three times a day (TID). He subsequently stopped using tobacco and his depression is in remission.

Finally, a 51 year old gentleman with NYSHA Class I CHF-TNF was 303 pg/ml before bupropion was started for depression and 7 pg/ml on 150 mg TID. This low TNF has been maintained for more than a year on bupropion.

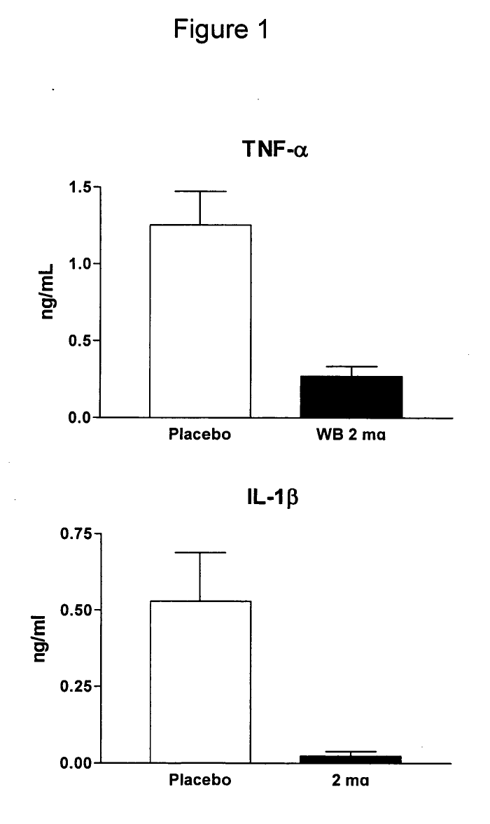

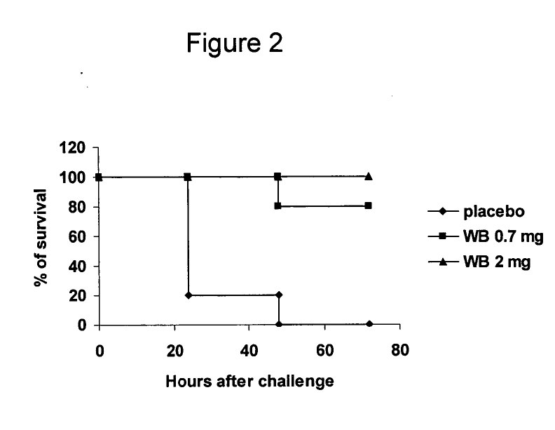

TNF is a member of a family of cytokines which also includes LIF , CNTF, Oncostatin M, ILI1, and CT-1. TNF-alpha is produced by many different cell types including stimulated monocytes, fibroblasts, and endothelial cells. Macrophages, T-cells and B-lymphocytes, granulocytes, smooth muscle cells, eosinophils, chondrocytes, osteoblasts, mast cells, glial cells,and keratinocytes also produce TNF-alpha after stimulation. Physiological stimuli for the synthesis of TNF-alpha include IL-1, bacterial endotoxins, TNF, PDGF , and Oncostatin M. We investigated the role of TNF-alpha in the host defense mechanism. Mice were treated with lipopolysaccharide (LPS) to mimic “sepsis” and to increase the circulating levels of TNF-alpha. The administration of buproprion decreased levels of TNF-alpha (FIG. 1, top) and significantly increased the survival time of mice subsequent to challenge (FIG. 2).

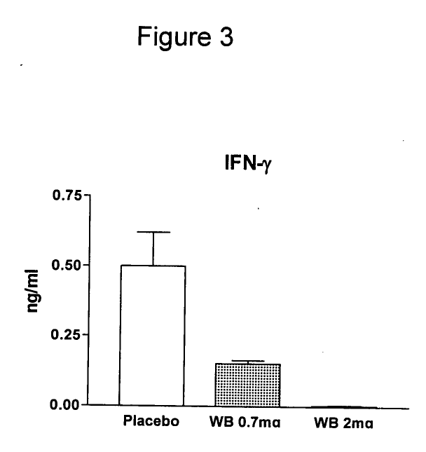

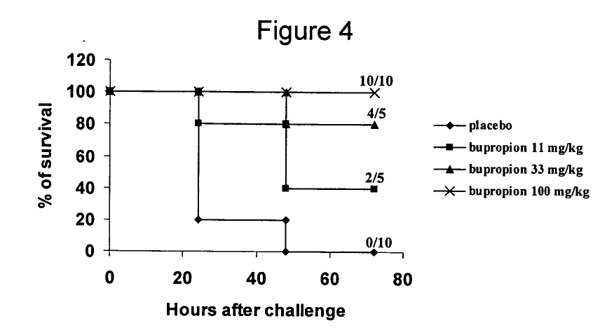

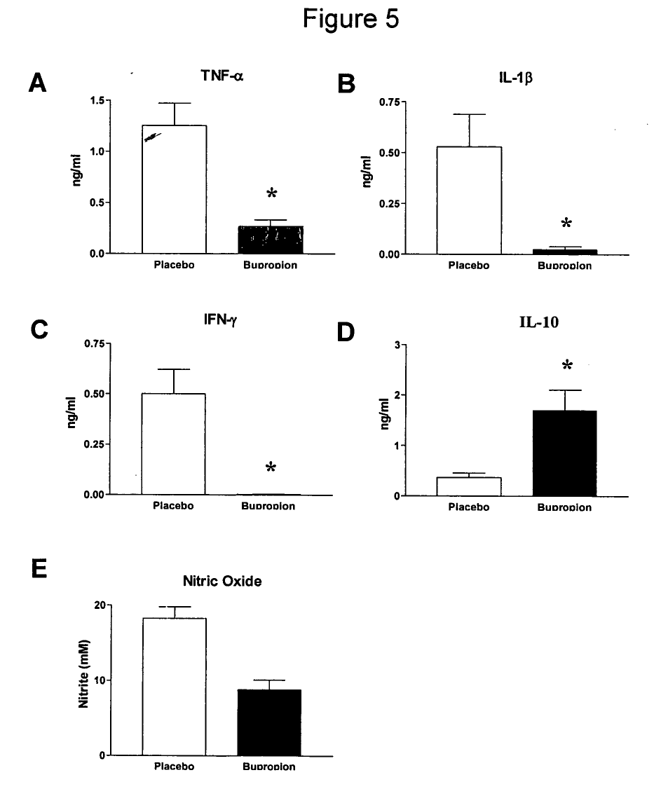

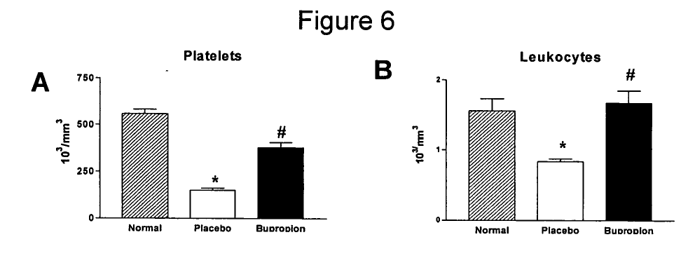

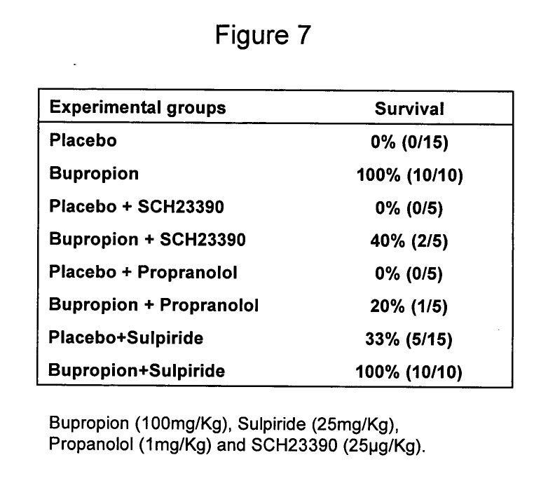

FIG. 1 is a bar graph depicting TNF-alpha levels (top) and IL-1beta levels in mice treated with a placebo or with bupropion hydrochloride ((+)-1-(3-chlorophenyl)-2-[(1,1-dimethylethyl)amino]-1-propanone hydrochloride) (WB). FIG. 2 is a line graph depicting survival curves for mice treated with a placebo or with increasing amounts of bupropion hydrochloride ((+)-1-(3-chlorophenyl)-2-[(1,1-dimethylethyl)amino]-1-propanone hydrochloride) (WB). FIG. 3 is a bar graph depicting IFN-gamma levels in mice treated with a placebo or with increasing amounts of bupropion hydrochloride ((+)-1-(3-chlorophenyl)-2-[(1,1-dimethylethyl)amino]-1-propanone hydrochloride) (WB). FIG. 4 is a line graph depicting Bupropion protection of mice against a lethal challenge of LPS. Male BALB/c mice were injected with 11, 33 or 100 mg/kg of bupropion or with placebo i.p. in saline 1 h before challenge with 600 μg of E. coli LPS (Sigma, St. Louis, Mo.) by i.p. route. Mice were sacrificed 90 min for detection of cytokines in the serum by sandwich ELISA. Values represent the means ±SEM of 5 mice per group. * P<0.05. FIG. 5 provides bar graphs depicting the effects of bupropion treatment in cytokine production in mice challenged with LPS. Groups of BALB/c mice were injected with bupropion or with placebo i.p. in saline 1 h before challenge with 600 μg of LPS (DL-100) by i.p. route. Bupropion significantly decreased levels of pro-inflammatory cytokines TNF, interleukin-1 beta (Il-1 beta), and interferon-gamma (INF-gamma), decreased with a trend toward significance the inflammatory mediator nitric oxide (NO), and had significantly increased levels of the anti-inflammatory (in this situation) Th2 cytokine Il-10. *P<0.05. FIG. 6 is a bar graph depicting normalization of platelet and leukocyte levels by bupripion treatment. Groups of BALB/c mice were injected with bupropion 100 mg/kg (Wellbutrin, Glaxo) or with placebo i.p. in saline 1 h before challenge with 600 μg of LPS (DL-100) by i.p. route. Platelet and leukocyte numbers were evaluated in peripheral blood samples collected 4 h after LPS challenge in an automated cell counter (Bayer). * P<0.05 placebo compared with normal levels; # P<0.05 bupropion compared with placebo. In a shock often platelet levels drop precipitously due to intravascular coagulation and leukocyte also fall secondary to TNF-induced apoptosis. The normalization by bupropion is consistent with the other effects of bupropion in this model. FIG. 7 is a table providing the results of the survival rates for animals treated with bupropion.

The data presented herein shows that the commonly used antidepressant bupropion (Wellbutrin®, GlaxoSmithKline) lowers levels of pro-inflammatory cytokines such as tumor necrosis factor-alpha (TNF-alpha) in a mouse model of TNF pathology, and protects mice challenged with a lethal dose of lipopolysaccharide (LPS). These effects of bupropion were abrogated by beta-adrenergic and dopamine (D1) receptor antagonists. Bupropion may thus be a useful medication in Crohn's disease and other TNF associated diseases with the ability to tap into deep and fundamental endogenous anti-inflammatory mechanisms.

Crohn's disease (CD), rheumatoid arthritis and a growing number of other diseases are associated with increased levels tumor necrosis factor-alpha (TNF) (Ghezzi and Cerami, Tumor necrosis factor as a pharmacological target. Methods Mol. Med. 98, 1-8, 2004). In these diseases current treatments such as salicylates, azathioprine, methotrexate and prednisone have often limited efficacy and can have severe side-effects with long-term use (Rutgeerts, Conventional treatment of Crohn's disease: objectives and outcomes. Inflamm. Bowel Dis. 7 Suppl 1, S2-8, 2001). Although anti-TNF and anti-TNF receptor antibodies have shown efficacy in TNF associated diseases, they occasionally provoke an immune response against themselves, or can cause autoimmune syndromes, thereby limiting their use (Brookes and Green, Maintenance of remission in Crohn's disease: current and emerging therapeutic options. Drugs 64, 1069-1089, 2004). New treatments for CD and other TNF diseases are needed. Cases of clinical remission of CD in patients taking the commonly used antidepressant bupropion (Wellbutrin®, GlaxoSmithKline) for depression or as an aide to smoking cessation were reported (Kast and Altschuler, Remission of Crohn's disease on bupropion, Gastroenterology 121, 1260-1261, 2001), and initial findings that bupropion lowers circulating TNF levels in humans indicate a possible immunomodulatory effect of this drug (Altschuler and Kast, Bupropion (Wellbutrin) lowers tumor necrosis factor-alpha (TNF) levels: implications for disease treatment, Soc. Neuro. Sci. Abstrs. 105.8, 2003).

The present data indicates that bupropion treatment reduces the levels of TNF and other pro-inflammatory cytokines and protects mice challenged with a lethal dose of lipopolysaccharide (LPS, Sigma, St. Louis, Mo., USA). These effects of bupropion were blocked by beta-adrenergic and dopamine (D1) receptor antagonists. Our results demonstrate that bupropion has a potent immunomodulatory action in vivo.

To study the immunomodulatory effects of bupropion we used a mouse model in which TNF production is induced by stimulation with lipopolysaccharide (LPS) (Bozza et al, The PACAP-type I receptor agonist maxadilan from sand fly saliva protects mice against lethal endotoxemia by a mechanism partially dependent on IL-10, Eur. J. Immunol. 28, 3120-3127, 1998). BALB/c mice were injected intraperitoneally (i.p.) with bupropion or placebo 1 h prior to i.p. injection of LPS (600 ug/mouse).

Male, 6 weeks old BALB/c mice were used in LPS-induced shock experiments. All mice were raised and maintained at the animal facilities at the Goncalo Moniz Research Center-FIOCRUZ, and provided with rodent diet and water ad libitum. Animal numbers in experimental groups were the minimum necessary to permit statistical analysis. All animals were sacrificed under anaesthesia.

Groups of mice were injected intraperitoneally (i.p.) with a dose of 600 μg of LPS (from Escherichia coli serotype 0111 :B4, Sigma, St. Louis, Mo., USA) in saline (previously determined as LD 100) 1 h after injection of placebo or bupropion. In some experiments, groups of mice received i.p. injection of propranolol (1 mg/Kg), SCH23390 (25 μg/kg) and sulpiride (25 mg/Kg) 1 h prior to injection of bupropion. Mice were monitored daily for 4 days. To determine serum TNF, IL-1 beta and IL-10 levels, mice were sacrificed 90 min after injection of LPS and bled by axilar vein puncture. Levels of IFN-gamma and nitric oxide were assayed using sera of mice sacrificed 4 h after LPS injection. Platelet and leukocyte cell counts were performed using blood collected with EDTA after 4 h of LPS challenge, using an automated cell counter (ADVIA 60, Bayer).

TNF, IL-1beta, IFN-gamma and IL-10 concentrations in serum samples or in supernatants from macrophage cultures were determined by enzyme-linked immunosorbent assay (ELISA) using antibody pairs and recombinant cytokines from R&D systems (Minneapolis, Minn., USA), according to the manufacturer's instructions. After incubation with streptoavidin-peroxidase conjugate (Sigma), the reaction was developed using 3,3′, 5,5′-tetramethylbenzidine (TMB) peroxidase substrate and read at 450 nm. The serum concentration of nitrite (NO2₋) was determined by the Griess reaction, as an indicator of nitric oxide production (Green et al., Analysis of nitrate nitrite and [¹⁵N] nitrite in biological fluids. Anal. Biochem 126, 131-138, 1982). Nitrate was reduced to nitrite by an enzymatic process previously described (Ding et al., Release of reactive nitrogen intermediates and reactive oxygen intermediates from mouse peritoneal macrophages, Comparison of activating cytokines and evidence for independent production, J. Immunol. 141, 2407-2412, 1988). The absorbance of the reaction product at 540 nm was measured using an ELISA reader. The NO2⁻ concentration was determined by using sodium nitrite as standard. Data were analyzed using Student's t test, one-way ANOVA or Newman-Keuls multiple comparison test, using Graph Pad Prism 3.0 software. Differences were considered significant when P<0.05.

Levels of the pro-inflammatory cytokines TNF, interleukin-lbeta (IL-1beta) and interferon-gamma (IFN-gamma) and in sera of mice pre-treated with 100 mg/kg of bupropion were significantly reduced in comparison with those of placebo treated mice after LPS challenge (FIG. 5A-C). In contrast, levels of the antiinflammatory cytokine IL-10 were significantly raised compared with placebo treated mice (FIG. 5D). The production of nitric oxide, stimulated by TNF and IFN-gamma and blocked by IL-10, is about four-fold reduced in bupropion treated mice compared to placebo treated mice (FIG. 5E).

Furthermore, bupropion mediated survival of the mice in the LPS-induced shock model in a dose dependent manner (FIG. 4). Signs of endotoxemia, such as diarrhea, immobility, lethargy, piloerection and shivering were milder or absent in mice treated with 100 mg/kg of bupropion, than in placebo treated mice. TNF activates the coagulation system causing platelet aggregation and may result in disseminated intravascular coagulation in LPS-induced shock, hypoxia, failure of multiple organs and death. Consistent with survival and protection from shock by bupropion, platelet counts 4 h after LPS challenge in bupropion-treated mice were significantly higher than placebo treated mice and similar to those of normal mice (FIG. 6A), correlating with the decreased TNF production observed in these mice. In shock and other situations TNF can cause apoptosis of leukocytes. Again, correlating with decreased TNF levels in bupropion treated mice compared with placebo treated mice, bupropion treated mice had higher leukocyte levels (FIG. 6B).

Bupropion is thought to be dopamine and norepinephrine reuptake inhibitor (Feighner, Mechanism of action of antidepressant medications, J. Clin. Psychiatry 60 Suppl 4, 4-11, 1999). Consistent with this we found that the non-selective beta-adrenergic receptor antagonist propranolol (Propranolol Chloridate, TEUTO) and the D1 selective dopamine receptor antagonist SCH23390 (Sigma, St. Louis, Mo., USA) were able to partially block bupropion mediated survival (FIG. 7). Neither of these compounds alone was able to protect mice from a lethal challenge with LPS. The D2 antagonist sulpiride (Equilid®, Aventis) alone, however, mediated partial protection in mice submitted to the endotoxic shock, and did not interfere with the protection induced by bupropion (FIG. 7).

The data provided herein indicate that bupropion lowers inflammatory cytokines by acting through the beta-adrenergic and D1 receptors, perhaps by increased intracellular cAMP causing decreased elaboration of inflammatory cytokines (Koshiba et al., Modification of cytokine milieu by A2A adenosine receptor signaling—possible application for inflammatory diseases, Nucleosides Nucleotides Nucleic Acids 23, 1101-1106, 2004). The partial protection against endotoxic shock afforded by sulpiride may be due to the negative coupling of D2 receptors to cAMP, so that antagonism at the D2 receptor leads, like stimulation of the beta adrenergic and D1 receptors, to increase cAMP (Vallar and Meldolesi, Mechanisms of signal transduction at the dopamine D2 receptor, Trends Pharmacol. Sci. 10, 74-77, 1989).

These in vivo results with bupropion are in good concordance with previous in vitro and in vivo work with dopamine and beta-adrenergic agonists and antagonists: Haskó and colleagues (Haskó et al., Modulation of lipopolysaccharide-induced tumor necrosis factor alpha and nitric oxide production by dopamine receptor agonists and antagonists in mice, Immunol. Lett. 49, 143-147, 1996) found that in vivo in a similar LPS-induced model as ours the non-selective dopamine agonist bromocryptine as well as sulpiride lowered TNF levels. Facchinetti and colleagues (Facchinetti et al., Dopamine inhibits responses of astroglia-enriched cultures to lipopolysaccharide via a beta-adrenoreceptor-mediated mechanism, J. Neuroimmunol. 150, 29-36, 2004) found that the addition of dopamine, noradrenaline or the non-selective beta-adrenergic agonist isoproteronol to in vitro cultures enriched in astrocytes significantly blunted LSP-stimulated TNF production. Various cell types of the immune system express dopamine and beta-adrenergic receptors, such as lymphocytes, natural killer cells and macrophages (McKenna et al., Dopamine receptor expression on human T- and B lymphocytes, monocytes, neutrophils, eosinophils and NK cells: a flow cytometric study. J. Neuroimmunol, 132, 34-40, 2002), a fact that opens the possibility of bupropion of a direct or indirect action in cytokine regulation through beta-adrenergic receptors.

The data demonstrate a potent immunomodulatory activity of bupropion, indicating that its association with remission of autoimmune diseases may be in fact due to its effects on immuno-inflammatory responses. For example, the present data demonstrate that bupropion-induced inhibition of TNF levels in a subject can be correlated with a reduction in symptoms associated with Crohn's disease and other TNF-related disorders such as rheumatoid arthritis, multiple sclerosis, glaucoma and congestive heart disease. The present data demonstrate that the administration of bupropion hydrochloride ((+)-1-(3-chlorophenyl)-2-[(1,1-dimethylethyl)amino]-1 propanone hydrochloride) to a subject inhibits TNF levels and alleviates symptoms associated with increased levels of TNF expression. Accordingly, the data provided herein support methods of inhibiting the physiological activity of TNF without causing beta-adrenergic receptor down-regulation in a subject by administering the subject to an effective dose of bupropion.

As noted above, experimental data is provided showing that TNF-alpha levels decrease in mice treated with bupropion hydrochloride ((+)-1-(3-chlorophenyl)-2-[1,1-dimethylethyl)amino]-1-propanone hydrochloride) as opposed to those levels treated with a placebo (FIG. 5A). The present data also demonstrate that IL-1β levels decrease in mice treated with bupropion hydrochloride ((+)-1-(3-chlorophenyl)-2-[1,1-dimethylethyl)amino]-1-propanone hydrochloride), in contrast to those treated with a placebo (FIG. 5B). Similar data is provided in FIG. 4 which demonstrates that mice treated with increasing amounts of bupropion hydrochloride ((+)-1-(3-chlorophenyl)-2-[1,1-dimethylethyl)amino]-1 propanone hydrochloride showed increased survival rates, in contrast to those treated with a placebo in a test that mimics sepsis. In addition, FIG. 5C demonstrates that IFN-gamma levels decrease in mice treated with increasing amounts of bupropion hydrochloride ((+)-1-(3-chlorophenyl)-2-[1,1-dimethylethyl)amino]-1 propanone hydrochloride), in contrast to those treated with a placebo.

Applicants have shown, as fully described throughout the specification, that the present methods are not only useful for treating Crohn's disease, but also for treating other conditions associated with expression of TNF, such as primary biliary cirrhosis (PBC) associated fatigue symptoms (Altschuler and Kast, Buprion for Fatigue and as a Tumor Necrosis Factor-alpha Lowering Agent in Primary Biliary Cirrhosis, Medical Hypotheses, 64:118-119 2005), loss of bone density associated with Crohn's disease (Kast and Altschuler, Bone Density Loss in Crohn's Disease: Role of TNF and Potential for Prevention by Bupropion, Gut, 53:1056, 2004), multiple myeloma (Kast and Altschuler, Combination of Bupropion, Paroxetine and Quetiapine as Adjuvant for Multiple Myeloma, Medical Hypotheses, 62:817-818, 2004), Blau syndrome (Altschuler and Kast, Bupropion for Blau Syndrome, Medical Hypotheses, 62:297-298, 2004), hepatitis B viral infection (Kast and Altschuler, Tumor Necrosis Factor-alpha in Hepatitis B: Potential Role for Bupropion, Journal Of Hepatology 39:131-132, 2003), psoriasis and atopic dermatitis (Altschuler and Kast, Bupropion in Psoriasis and Atopic Dermatitis: Decreased Tumor Necrosis Factor-alpha?, Psychosomatic Medicine 65:719, 2003). In addition, Crohn's disease and B cell chronic lymphocytic leukemia (CLL) share a common link in their pathologic mechanisms. Lymphocytes in both diseases fail to undergo apoptosis and die properly. That failure is partly due to increased signaling by tumor necrosis factor (TNF)-alpha, and their respective pathologies directly follow from this apoptosis failure. Bupropion is a commonly used generic antidepressant in clinical use for over a decade, and early evidence indicates it lowers TNF levels (Kast and Altschuler, Anti-apoptosis function of TNF-alpha in chronic lymphocytic leukemia: lessons from Crohn's disease and the therapeutic potential of bupropion to lower TNF-alpha, Arch Immunol Ther Exp (Warsz), 53(2): 143-7, 2005). In view of this information, and in light of the data described in the present specification, bupropion can be used to lower TNF levels and slow CLL disease course.

The data described herein, and in the aforementioned publications incorporated herein by reference, provide a basis for treating diseases by inhibiting the physiological activity of TNF without causing beta-andrenergic receptor down-regulation in a subject by administering to the subject an effective dose of bupropion. The data provided herein demonstrates that bupropion-induced inhibition of TNF levels in a subject can be correlated with a reduction in symptoms associated with Crohn's disease. The data also demonstrates and describes the use of bupropion to treat a wide variety of disorders associated with increased levels of TNF.

Methods for treating TNF-associated disorders have been described with reference to several embodiments. Important properties and characteristics of the described embodiments are illustrated in the text and in the accompanying drawings. While the invention has been described in conjunction with these embodiments, it should be understood that the invention it is not intended to be limited to these embodiments. On the contrary, it is intended to cover alternatives, modifications, and equivalents as may be included within the spirit and scope of the invention as defined by the appended claims. Accordingly, the examples set forth above, and the accompanying drawings, are provided to give those of ordinary skill in the art with a complete disclosure and description of how to make and use the preferred embodiments of the compositions, and are not intended to limit the scope of what the inventors regard as their invention. All patents and publications mentioned in the specification are indicative of the levels of skill of those skilled in the art to which the invention pertains. All references cited in this disclosure are incorporated by reference to the same extent as if each reference had been incorporated by reference in its entirety individually.

Modifications of the above-described modes for carrying out the invention that are obvious to persons of skill in the art are intended to be within the scope of the following claims. While the present invention has now been described in terms of certain preferred embodiments, and exemplified with respect thereto, one skilled in the art will readily appreciate that various modifications, changes, omissions and substitutions may be made without departing from the spirit thereof. It is intended, therefore, that the present invention be limited solely by the scope of the following claims.

What is claimed is:

1. A method of inhibiting a physiological activity of tumor necrosis factor (TNF) without causing beta-andrenergic receptor down-regulation, in a subject, the method comprising:
administering to the subject an effective dose of bupropion hydrochloride ((+)-1-(3-chlorophenyl)-2-[(1,1-dimethylethyl)amino]-1-propanone hydrochloride), or analogues thereof.

2. The method of claim 1, wherein the physiological activity of TNF is associated with Crohn's disease, rheumatoid arthritis, psoriasis, IgA nephropathy, anemia, myelodysplasia, appendicitis, peptic, gastric and duodenal ulcers, peritonitis, pancreatitis, pseudomembranous colitis, acute ulcerative colitis, chronic ulcerative colitis and ischemic colitis, diverticulitis, epiglottitis, achalasia, cholangitis, cholecystitis, hepatitis, nosicomial infection, inflammatory bowl disease, enteritis, Whipple's disease, diabetes, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pelvic inflammatory disease, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, urticaria, warts, wheals, vasulitis, cardiovascular disease, angiitis, endocarditis, arteritis, atherosclerosis, thrombophlebitis, pericarditis, myocarditis, myocardial ischemia, periarteritis nodosa, rheumatic fever, rheumatoid arthritis, Alzheimer's disease, coeliac disease, congestive heart failure, adult respiratory distress syndrome, meningitis, encephalitis, multiple sclerosis, cerebral infarction, cerebral embolism, Guillane-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, Sjogren's syndrome, myasthenia gravis, thryoiditis, systemic lupus erythematosus, lupus erythematosus, Addison's disease, pernicious anemia, Goodpasture's syndrome, Behcets's syndrome, allograft rejection, graft-versus-host disease, Type I diabetes, ankylosing spondylitis, Berger's disease, Type I diabetes, ankylosing spondylitis, spinal cord injury, Retier's syndrome, Graves disease, or Hodgkins disease.

3. The method of claim 1, wherein the effective dose is in the range of 15 mg twice a day to 150 mg three times a day for an adult patient.

4. The method of claim 1, wherein the subject is a mammal.

5. The method of claim 4, wherein the mammal is a mouse.

6. The method of claim 5, wherein the mammal is a primate.

7. The method of claim 6, wherein the primate is a human.

8. A method for inhibiting the release of tumor necrosis factor (TNF) from a mammalian cell, the method comprising contacting the cell with bupropion hydrochloride ((+)-1-(3-chlorophenyl)-2-[(1,1-dimethylethyl)amino]-1-propanone hydrochloride), or analogues thereof, in an amount effective to inhibit the release of tumor necrosis factor (TNF).

9. The method of claim 8, wherein the cell is in a patient suffering from, or at risk for, a disease or disorder mediated by a tumor necrosis factor (TNF)—associated cascade.

10. A method for decreasing the circulating level of tumor necrosis factor (TNF) in a subject, the method comprising administering to the subject an effective dose of bupropion hydrochloride ((+)-1-(3-chlorophenyl)-2-[(1,1-dimethylethyl)amino]-1-propanone hydrochloride), or analogues thereof, said administering being in an amount effective to decrease the circulating level of tumor necrosis factor (TNF) in the subject.

11. A method for ameliorating symptoms resulting from a disorder associated with tumor necrosis factor (TNF) without causing beta-andrenergic receptor down-regulation, the method comprising administering to the subject an effective dose of bupropion hydrochloride ((+)-1-(3-chlorophenyl)-2-[(1,1-dimethylethyl)amino]-1-propanone hydrochloride), or analogues thereof, said administering being in an amount effective to decrease the circulating level of tumor necrosis factor (TNF) in the subject thereby ameliorating the symptoms of the disorder.

12. The method of claim 11, wherein the disorder is selected from the group consisting of Crohn's disease, rheumatoid arthritis, psoriasis, IgA nephropathy, anemia, myelodysplasia, appendicitis, peptic, gastric and duodenal ulcers, peritonitis, pancreatitis, pseudomembranous colitis, acute ulcerative colitis, chronic ulcerative colitis and ischemic colitis, diverticulitis, epiglottitis, achalasia, cholangitis, cholecystitis, hepatitis, nosicomial infection, inflammatory bowl disease, enteritis, Whipple's disease, diabetes, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pelvic inflammatory disease, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, urticaria, warts, wheals, vasulitis, cardiovascular disease, angiitis, endocarditis, arteritis, atherosclerosis, thrombophlebitis, pericarditis, myocarditis, myocardial ischemia, periarteritis nodosa, rheumatic fever, rheumatoid arthritis, Alzheimer's disease, coeliac disease, congestive heart failure, adult respiratory distress syndrome, meningitis, encephalitis, multiple sclerosis, cerebral infarction, cerebral embolism, Guillane-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, Sjogren's syndrome, myasthenia gravis, thryoiditis, systemic lupus erythematosus, lupus erythematosus, Addison's disease, pernicious anemia, Goodpasture's syndrome, Behcets's syndrome, allograft rejection, graft-versus-host disease, Type I diabetes, ankylosing spondylitis, Berger's disease, Type I diabetes, ankylosing spondylitis, spinal cord injury, Retier's syndrome, Graves disease, and Hodgkins disease.

13. The method of claims 1, 8, 10, or 11, wherein the bupropion hydrochloride ((+)-1-(3-chlorophenyl)-2-[(1,1-dimethylethyl)amino]-1-propanone hydrochloride), or analogues thereof, is administered in conjunction with a steroid or an antibody directed against tumor necrosis factor (TNF).

14. A method for treating a tumor necrosis factor (TNF) associated disease without causing beta-andrenergic receptor down-regulation, comprising administering bupropion hydrochloride ((+)-1-(3-chlorophenyl)-2-[(1,1-dimethylethyl)amino]-1-propanone hydrochloride), or analogues thereof, in an effective dose to alleviate or eliminate TNF associated disease symptoms and pathology; and monitoring the patient's response wherein the dose is adjusted according to degree of alleviation and elimination of the symptoms and pathology.

15. The method of claim 14, wherein monitoring the patient's response includes monitoring the patient's Crohn's disease activity index (CDAI) level.

16. The method of claim 14, wherein monitoring the patient's response includes monitoring the patient's circulating level of TNF.

17. The method of claim 14, wherein the tumor necrosis factor (TNF) associated disease is rheumatoid arthritis.

18. The method of claim 14, wherein the tumor necrosis factor (TNF) associated disease is psoriasis.

19. The method of claim 14, wherein the tumor necrosis factor (TNF) associated disease is IgA nephropathy.

20. The method of claim 14, wherein the tumor necrosis factor (TNF) associated disease is anemia.

21. The method of claim 14, wherein the tumor necrosis factor (TNF) associated disease is myelodysplasia.
